(19) United States
(12) Patent Application Publication
(10) Pub. No.: US 2011/0040271 A1
(43) Pub. Date: Feb. 17, 2011

(54) Urine Catchment Device

(76) Inventor: Jane Rogers, Deltona, FL (US)

Correspondence Address: Premier Law Group, PLLC, 3975 University Drive, SUITE 330, Fairfax, VA, 22030, US

(21) Appl. No.: 12/855,150

(22) Filed: Aug. 12, 2010

Related U.S. Application Data
(60) Provisional application No. 61/233,223, filed on Aug. 12, 2009.

(51) Int. Cl. A61F 5/455

(52) U.S. Cl. 604/346

(57) ABSTRACT

An external, body worn urine catchment and drainage appliance for bedridden females, which cooperates with the natural contours of the human body. The urine catchment device comprises a receptacle which has external bulges or corresponding configurational features providing structure for cooperating with and engaging recesses of the anatomy and a discharge tube for discharging collected urine. Although upwardly open to the atmosphere in the operative position, the urine catchment device has structure for sealing against urine escaping past the receptacle as it flows into the receptacle.

### CROSS-REFERENCE TO RELATED APPLICATIONS

This application claims the benefit of priority from U.S. Provisional Patent Application Ser. No. 61/233,223 filed on 08-12-2009. The entire content of Provisional Patent Application Ser. No. 61/233,223 is incorporated herein by reference in its entirety.

### STATEMENT REGARDING FEDERALLY SPONSORED RESEARCH OR DEVELOPMENT

Not Applicable.

### FIELD OF THE INVENTION

The present invention is directed to a urine catchment and drainage appliance or device for bedridden females.

### BACKGROUND OF THE INVENTION

Women who are bedridden and/or are not easily or safely ambulatory must be attended to for safe disposal of urine. Various devices have been proposed to collect urine for subsequent disposal. These devices address the need for a device that can be held against the body in different ways. Some may rely upon adhesive or encirclement of the body for example. These devices may also include engagement elements that are adapted to maintain the subject device in an effective, stable position despite body movements, gravity, and other disturbing influences.

There exists a need for a body engaging urine catchment device which is more effective and easier to use than other known urine collection devices.

### SUMMARY OF THE INVENTION

The present invention is an external, body worn urine catchment and drainage appliance for such as bedridden females and women are not easily or safely ambulatory. The purpose of the appliance or device is to intercept and collect discharged urine for disposal, and to prevent contamination by urine of the body, clothing, a bed, and bedclothes. The device utilizes the natural configuration of the human body to engage the body of a bedridden female and is retained in operative position thereon. To this end, the novel urine catchment device comprises a receptacle which has external bulges or corresponding configurational features providing structure to cooperatively engage the recesses, curves, and convexities of the female user's anatomy. However, these anatomy engaging features do not require deep penetration for example of the vagina. Therefore, cooperation with the anatomy is such that a comfortable fit is achieved, while still improving on the ability of a collection device to remain in place on the body and entrapping discharged urine. In particular, the novel device accommodates lying on the side while sleeping.

Generally, the urine catchment device comprises a bowl of special configuration for collecting urine from the urethra, and a discharge tube for emptying. Although upwardly open to the atmosphere in the operative position, the urine catchment device includes a seal for contacting against the female body to prevent urine from leaking out of the receptacle as it flows into the receptacle. A foundation garment worn by the user or patient, such as briefs, diaper, etc., may assist in keeping the urine catchment device in place, but is not critical.

The receptacle may be drained through a collection tube which may be connected to a drainage conduit which is integral with the receptacle or bowl.

The urine catchment device eliminates any need for devices such as bedpans and absorbent apparel such as briefs or panties that are worn to absorb urination, although use of foundation garments may help in keeping the urine catchment device in place during movements while sleeping, for example. The urine catchment device does not require straps or other structure for encircling the human body or adhesives to assure retention thereon, although such features may optionally be provided if desired.

### BRIEF DESCRIPTION OF THE DRAWINGS

FIG. 1 is a top perspective view of a urine catchment device according to at least one aspect of the invention.

FIG. 2 is a top view of FIG. 1.

FIG. 3 is a top front perspective view of FIG. 1.

FIG. 4 is a side elevational view of FIG. 1.

FIG. 5 is an environmental side elevational view of a urine catchment device such as the urine catchment device of FIG. 1 shown in operative position on the body of a person using the urine catchment device.

FIG. 6 is a top plan view of FIG. 5, with panties or briefs rendered in broken lines.

FIG. 7 is a top plan line drawing of the urine catchment device of FIG. 1.

FIG. 7A shows a section view of the urine catchment device of FIG. 1.

FIG. 8 is a diagrammatic view of an alternative configuration of an opening for a urine catchment device according to the present invention.

FIG. 9 is a diagrammatic view of a further alternative configuration of an opening for a urine catchment device according to the present invention.

FIG. 10A is a side cross sectional view of a urine catchment device according to an aspect of the present invention.

FIG. 10B is a rear elevational view of FIG. 10A.

FIG. 11A is a side cross sectional view of a urine catchment device according to another aspect of the invention.

FIG. 11B is a side cross sectional view of a urine catchment device according to still another aspect of the invention.

FIG. 12 is a side cross sectional view of a urine catchment device according to a further aspect of the invention.

FIG. 13 is a side elevational view of a urine catchment device according to yet another aspect of the invention.

FIG. 14 is a side elevational view of a urine catchment device according to still another aspect of the invention.

FIG. 15 is a top plan view of a urine catchment device according to a further aspect of the invention.

FIG. 16 is a side perspective view of a urine catchment device according to a still further aspect of the invention.

FIG. 17 is a side elevational view of a urine catchment device according to another aspect of the invention.

FIG. 18 is a side elevational view of a urine catchment device according to still another aspect of the invention, with alternative positions of two members shown in broken lines.

FIG. 18A is a top view as shown in FIG. 2, but with spring spars added according an additional aspect of the invention.

FIG. 19 is a side elevational view of a urine catchment device according to an additional aspect of the invention.

FIG. 20 is a top plan view of a urine catchment device according to a further additional aspect of the invention.

FIG. 21 is a top plan view of a urine catchment device according to a still further aspect of the invention.

FIG. 22 is a top plan view of a urine catchment device according to another aspect of the invention.

FIG. 23 is a side elevational view of a urine catchment device according to a still further aspect of the invention.

### DETAILED DESCRIPTION

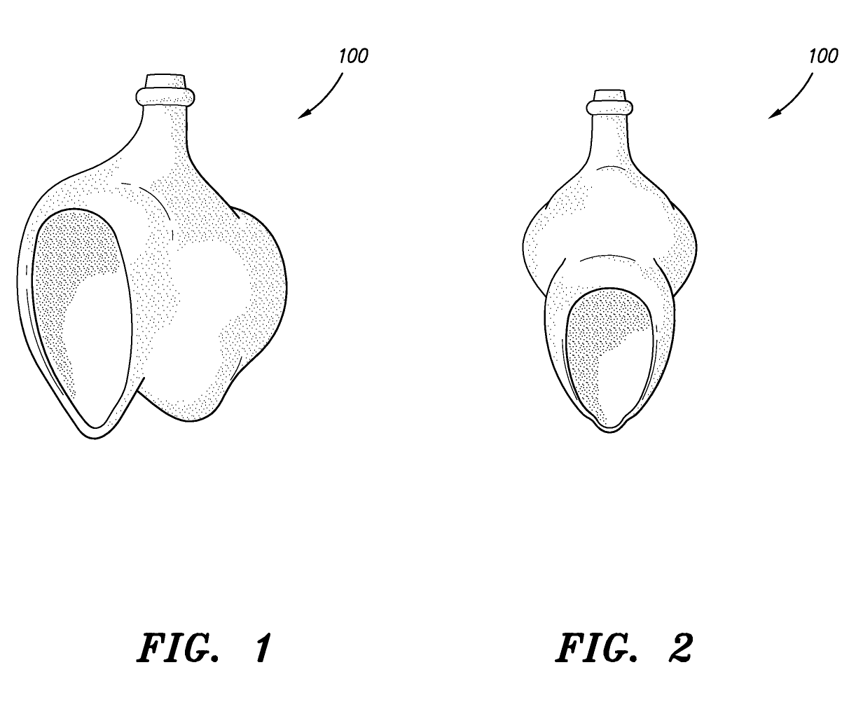

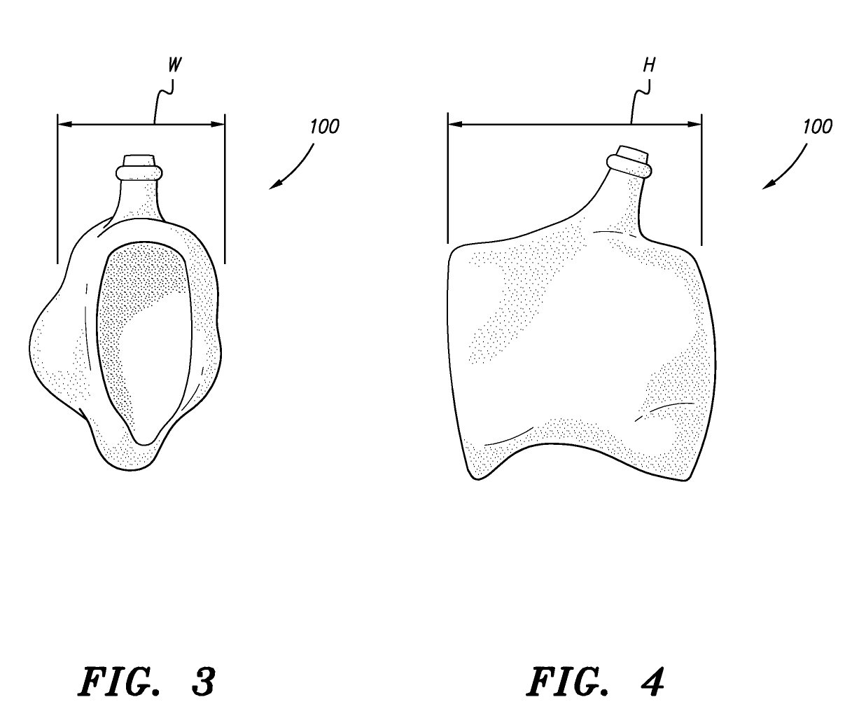

A urine catchment device 100 according to at least one aspect of the present invention is depicted in FIGS. 1-4, 7 and 10A. The urine catchment device 100 has a collection receptacle or bowl 120 bounded by a circumferential lateral wall 140 and a floor 160, the floor being located on the bottom of the device 100. The floor 160 may be flat so as to cooperate with a flat bed surface such as the top of a mattress (not shown). The bowl 120 defines a hollow interior chamber 125 and opposite right and left sides 130a and 130b. The terms “collection receptacle” and “bowl” are hereinafter regarded as equivalent terms. In addition, the terms “patient” and “female user” are regarded as equivalent terms.

The bowl 120 is in fluid communication with a discharge tube 180 having a fluid passageway 200 extending longitudinally therethrough, which terminates at an opening 220. The top of the lateral wall 140 forms a lip 240. The lip 240 defines an opening 400a of bowl 120. The opening 400a allows urine to be collected into the hollow interior chamber 125. In one embodiment the circumferential lateral wall 140, floor 160 and discharge tube 180 are manufactured as a single integrated structure.

It will be seen that a front bulge 260, a right side bulge 280, and a left side bulge 300 may be formed in the urine catchment device 100. More specifically, the circumferential lateral wall 140 defines front bulge 260, right side bulge 280, and left side bulge 300. The bulges 260, 280, and 300 are convexities for cooperatively engaging anatomical features of the body to assist in holding the urine catchment device 100 in operable position, as will be further detailed hereinafter.

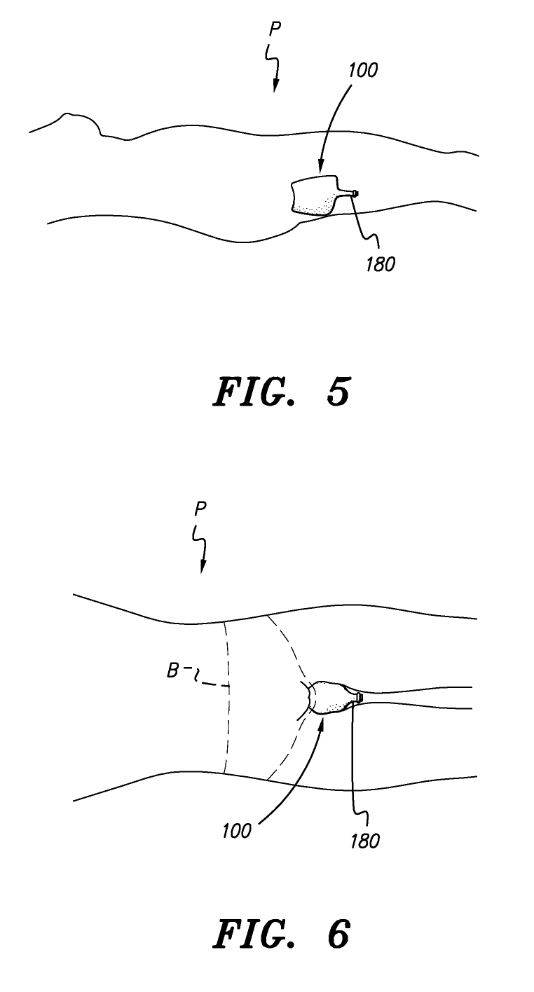

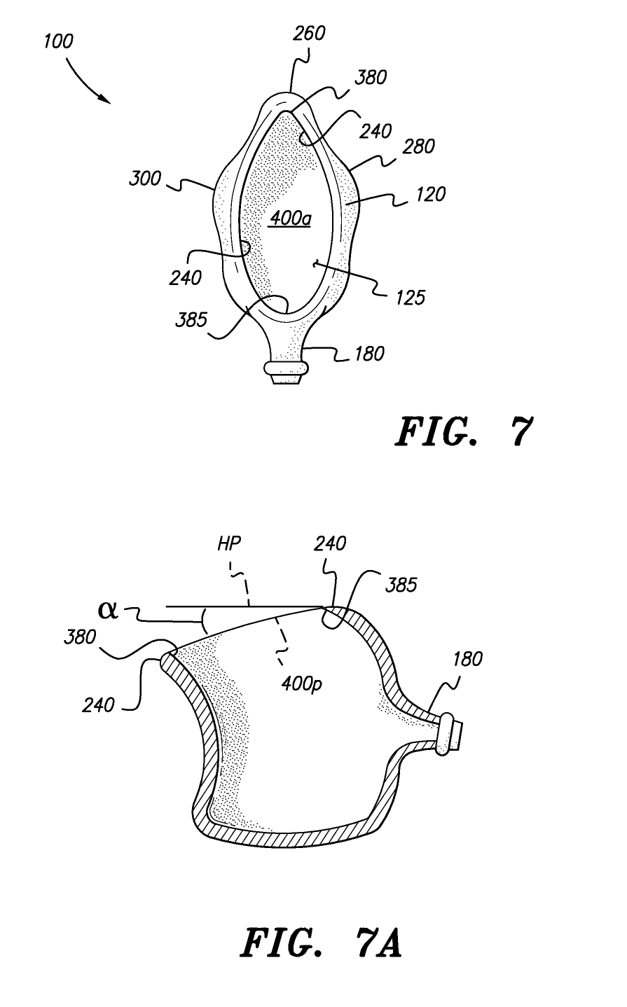

FIGS. 5 and 6 show the urine catchment device 100 in operable position installed on the body of a patient P, shown lying on her back, such as on a mattress (not shown). The urine catchment device 100 has been placed in abutment with the perineum just below the vagina. Although open at the top, the urine catchment device intercepts and retains a flow of urination in the bowl 120. Because of the precise placement of the urine catchment device, anatomical features such as the labia majora or the labia minora deflect urine so that the urine is not ejected in a forceful stream which would bypass the urine catchment device 100. However, as will be described hereinafter, structure for intercepting such forceful streams may be provided. Also, structural features of the urine catchment device 100 assure effective sealing at the interface between the body and the urine catchment device 100 so as to preclude entirely or almost entirely leakage past the urine catchment device 100 onto the mattress.

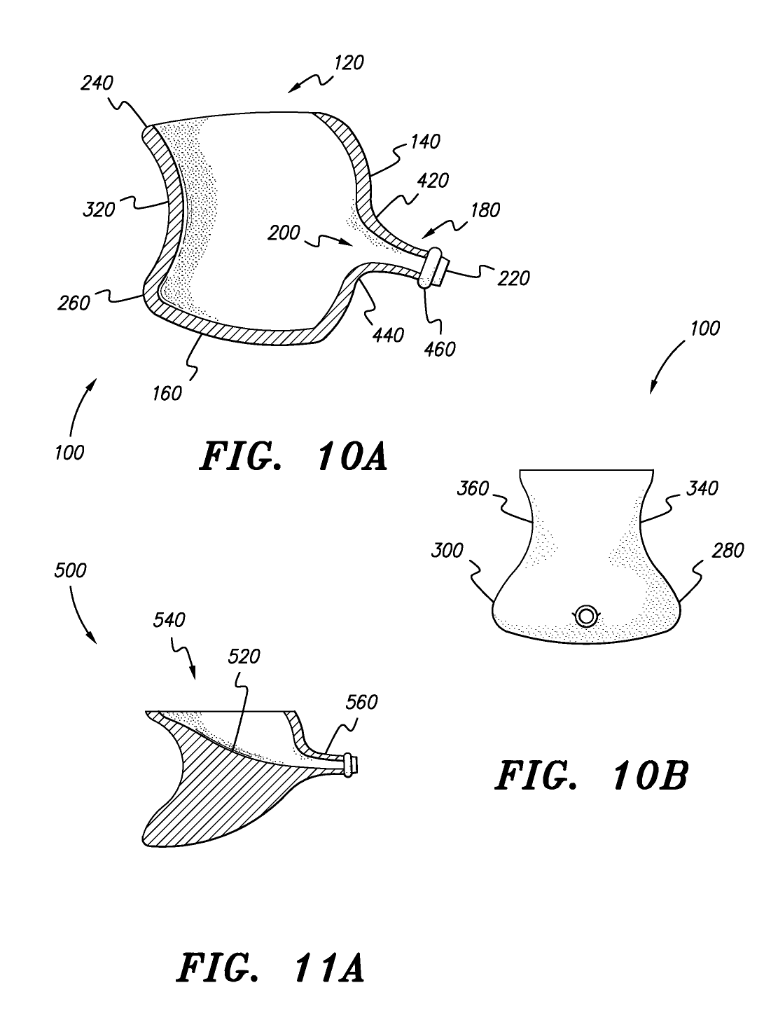

The urine catchment device 100 may be retained on the body in the following way. First, the urine catchment device 100 has projections such as the bulges 260, 280, and 300, which engage the body. The bulges 280, 300 engage inner and posterior thigh areas and into the swellings of inferior buttocks. Secondly, a garment such as a brief B or other pelvis hugging or surrounding garment may entrap the urine catchment device 100 between the body and the garment. The front surface 320 (see FIG. 10A) formed in the urine catchment device 100 is received in and is gripped by the inferior gluteal cleft, thereby preventing side to side motion and loss. It will be seen in FIG. 10A that the front surface 320 when viewed from the side is somewhat concave. Although other surfaces which contact the skin of the user to establish grasp may be textured in various ways to enhance frictional engagement of the skin, the frontmost portion of the front surface 320 may be smooth.

Referring specifically to FIG. 10A, curvature 420 and curvature 440 may cooperate with a garment such as the brief B to assist in retaining the urine catchment device 100 in place. These curvatures 420 and 440 may collectively form an arc that passes between the thighs in the midsagittal plane from posterior and inferior to or behind the anterior mons pubis to the posterior rump to establish a snug fit relative to the garment.

The lip 240 may establish a seal with the perineum, posterior to the genitals (i.e., just below the female user's vaginal area), and anterior to the anus. This seal does not fully close the receptacle 120 to the atmosphere; rather, it seals against escape of the urine by preventing it from flowing downwardly along the body of the patient or the exterior of the urine catchment device 100. The sides of the urine catchment device 100, as best seen in FIG. 10B, form concave recesses 340, 360 just above the bulges 280, 300, generally midway between the top and bottom of the urine catchment device 100. These concave recesses 340, 360 are shaped to compliment and engage the inside shape of the female user's thighs to enable the surfaces of the recesses 340, 360 to be fitted and comfortably gripped by the inner thighs of the female user to keep the device 100 in the operative position as shown in FIGS. 5 and 6.

The walls of the urine catchment device 100 at the recesses 340, 360 may bear texturing to enhance frictional grip of the skin. Texturing may take the form of bumps, wrinkles, or knurling, or other corresponding elements introduced into the otherwise smooth surface.

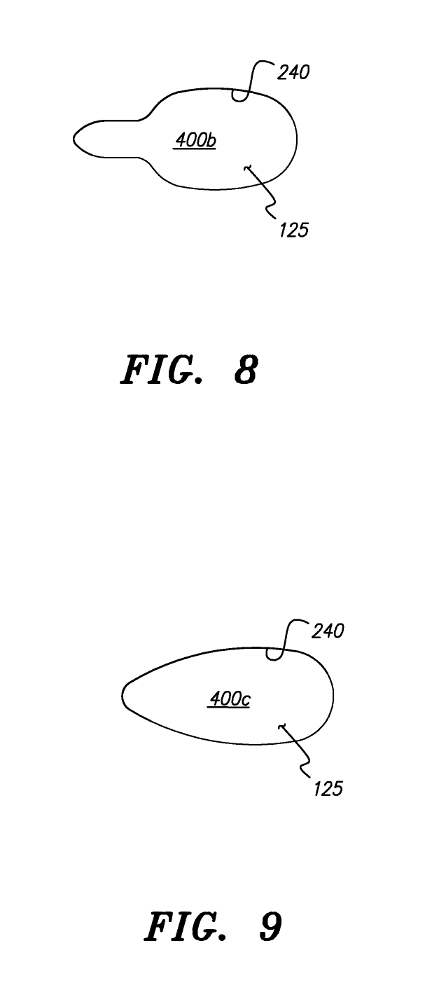

The sealing effect against the perineum, i.e., just below the female's vaginal orifice, when the female user is in the supine position, and overall fit of the urine catchment device 100 to the body may be enhanced by certain characteristics of the urine catchment device 100 apart from those already described. Referring particularly to FIG. 7, it will be seen that the lip 240 takes on a somewhat pointed and rounded configuration at the front end 380 thereof. This pointed front end 380 modifies the overall shape of the opening 400a bounded by the lip 240 from what would otherwise be an oval. The modified oval of FIG. 7 is not the only possible configuration of the opening. Other examples of corresponding openings 400b, 400c are seen in FIGS. 8 and 9, respectively.

Another characteristic which may assist in retaining the urine catchment device 100 to the body is flexifbility of the constituent material. While it is possible that a rigid material may be employed, it is also possible to use partially flexible materials and fully flexible plastic materials, and to form some parts of the device from one constituent material and other parts from other constituent materials. Suitable constituent materials include but are not limited to polymeric materials and silicone materials, alone or in combination. Flexibility may be imparted by materials which incorporate substances in granular or particle form, inflatable constructions, and constructions having bubble-like inclusions. A partially flexible material is one which has sufficient rigidity to hold its form when not subjected to external forces other than gravity, but which will yield or deform responsive to forces such as finger pressures. A fully flexible material is one which may spontaneously slump or otherwise not hold its form in response only to gravity. Of course, flexibility may be a function of wall thickness as well as of the selected constituent material.

As seen in FIG. 10A, the discharge tube 180 may be arranged to incline downwardly to assist flow of urine to an external device (not shown) for disposal. The external device may comprise a tube or hose or other device, or alternatively may comprise complementing conduits and conduits formed in complementing sections, at least one of which may be detachable and replaceable. Connection may be by friction, resilient grip, bayonet connection, and in other ways. It would be possible to engage the discharge tube 180 by inserting the end of a cooperating conduit into the opening 220. The discharge tube 180 may comprise one or more circumferential ridges 460 which enhances engagement of an external tube or hose or a specially designed connector or adapter (not shown) which may be slipped over the discharge tube 180.

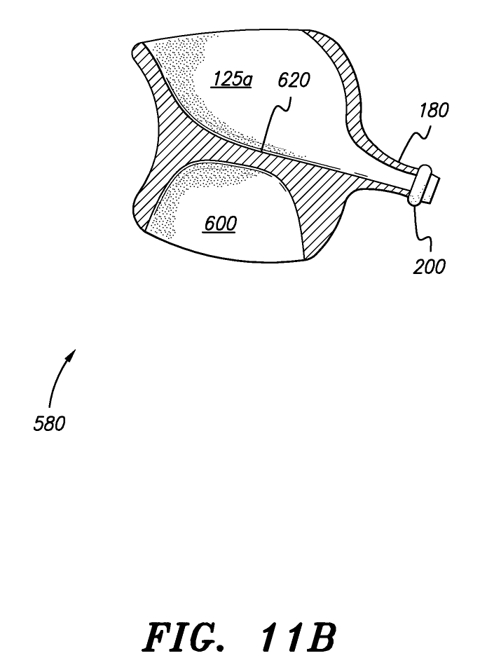

FIG. 11A depicts a urine catchment device 500 which may be generally similar in structure and function to the urine catchment device 100, with the following exception. The floor 520 of the urine collection receptacle 540 may slope towards a discharge tube labeled in FIG. 11A as 560. The sloping surface may be either straight or curved.

FIG. 11B depicts a urine catchment device 580, which is generally similar in structure and function to the urine catchment device 100 except that in the urine catchment device 580 there is an additional open hollow chamber 600. Hence urine catchment device 580 is a modified form of urine catchment device 100 having a chamber 125 (depicted by alpha-numeric label “125a”). The additional hollow chamber 600 is located below a floor 620. A discharge tube 180 is linked to chamber 125a such that collected urine can be discharged through opening 200 of discharge tube 180. The modified construction of urine catchment device 580 serves to conserve constituent material of and also helps to maintain flexibility of the urine catchment device 580.

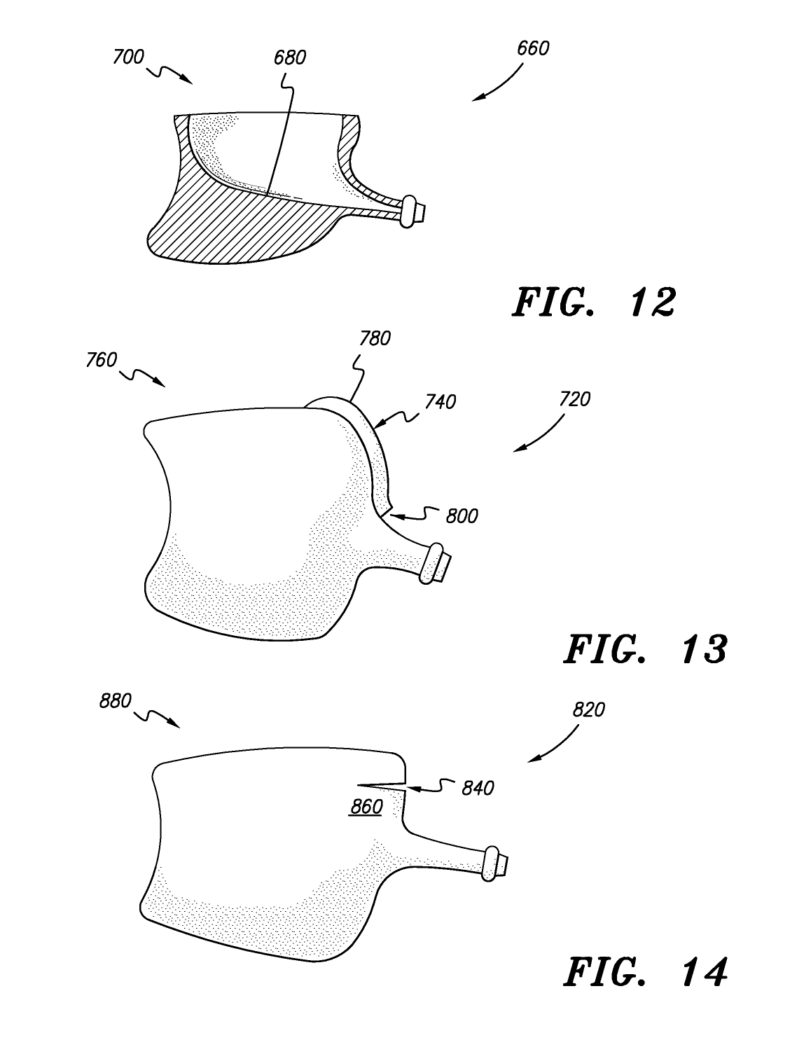

FIG. 12 shows a further example of a urine catchment device 660 which may be generally functionally and structurally similar to the urine catchment device 500 for example. However, in the urine catchment device 660, the floor 680 of the urine collection receptacle 700 can be curved as shown.

FIG. 13 shows a further example of a urine catchment device 720 which may be generally functionally and structurally similar to the urine catchment device 100 for example. However, the urine catchment device 720 may have an air passage 740 molded thereinto. The air passage 740 is provided for the purpose of relieving vacuum which may develop within the urine collection receptacle 760 should pressures below ambient air pressure arising from interaction with the patient's body force closed the otherwise open top of the urine catchment device or otherwise interfere with intended operation. As depicted, the air passage 740 may comprise a ridge 780 which is discernible as a discrete entity, having a proximal opening 800 for entry of air and a corresponding distal opening (not visible in FIG. 13) which allows air to flow into the urine collection receptacle 760. Of course, as an alternative to being contained within the ridge 780, the actual air passage may be molded into the wall of the urine catchment device 720 so as not to be discernible, or alternatively may comprise a raised open channel, or an open channel depressed into the surface of the urine catchment device 720. The top of the raised open channel, where used, may be notched to provide intake vacuum reliefs. The open channel may include several of these characteristics, and may vary in number and location from the examples actually illustrated and described herein. Not all of the possible permutations and combinations of an open channel or an equivalent thereof are shown.

FIG. 14 shows a urine catchment device 820 which may be generally functionally and structurally similar to the urine catchment device 100 for example, which provides another way to relieve vacuum. A slit 840 or other opening is formed in the rear wall 860 of the urine collection receptacle 880. It will be understood that venting can be provided with a combination of one or more slits and one or more vent openings.

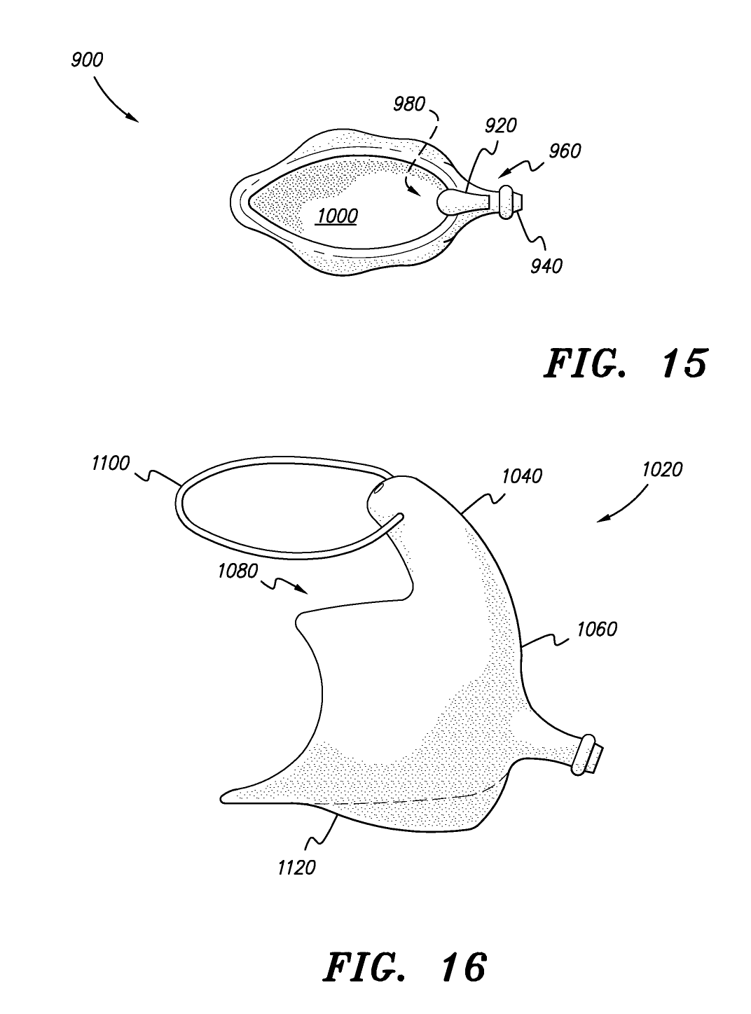

FIG. 15 shows a further example of a urine catchment device 900 which may be generally functionally and structurally similar to the urine catchment device 100 for example. However, in the urine catchment device 900, an air passage (not visible in FIG. 15) is formed in a projecting conduit 920 which may extend in a direction generally parallel to that of the discharge tube 940. The air passage may extend between an exposed proximal opening 960 and an exposed distal opening 980 which opens to the interior of the urine collection receptacle 1000.

FIG. 16 shows a still further example of a urine catchment device 1020 which may be generally functionally and structurally similar to the urine catchment device 100 for example. The urine catchment device 1020 may have a hood 1040, or alternatively stated, an extension of the rear wall 1060 of the urine collection receptacle 1080. The hood 1040 imposes an elevated barrier which may intercept and obstruct urine which might have been ejected as a jet from the body, or may intercept and obstruct splashing of urine contained within the urine collection receptacle 1080. It will be understood that the length of the hood 1040 can be any suitable length.

A loop 1100 may serve as a handle for holding and maneuvering the urine catchment device 1020, and may be fixed to the hood 1040 as shown in FIG. 16.

FIG. 16 also shows variations of the bottom surface 1120 of the urine collection receptacle 1080 which are possible. The bottom surface 1120 may incorporate a downwardly projecting bulge (rendered in solid line) if desired. Alternatively, the bottom surface 1120 may have a flat, straight configuration (represented in broken line) if desired. Still other configurations are possible.

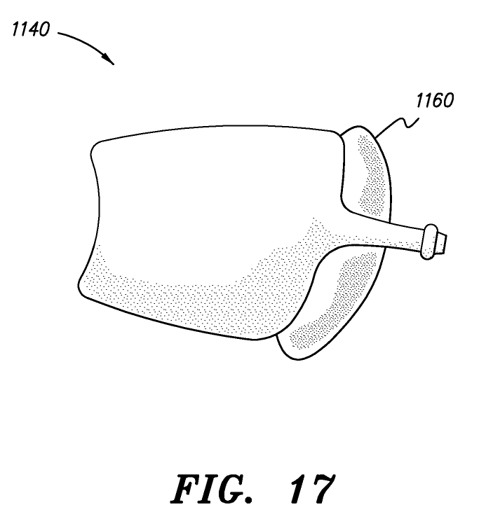

Referring now to FIG. 17, anchorage of a urine catchment device 1140 to the body of a patient may be enhanced by elastic or resilient engagement of the garment such as the briefs B (see FIG. 6). The urine catchment device 1140 provides a further example of a urine catchment device which may be generally functionally and structurally similar to the urine catchment device 100 for example. The urine catchment device 1140 may comprise a resilient or elastic member 1160, which may comprise a block of synthetic resin foam for example, and which projects outwardly from the urine catchment device 1140 proximate the discharge tube (such as the discharge tube 180 of FIG. 10A). The elastic member 1160 may be somewhat compressed between the garment and the urine catchment device 1140, and thereby assist in immobilizing the urine catchment device 1140. The elastic member 1160 may be integral with the urine catchment device 1140, or alternatively may comprise a physically separate member which of course may be readily attachable and detachable.

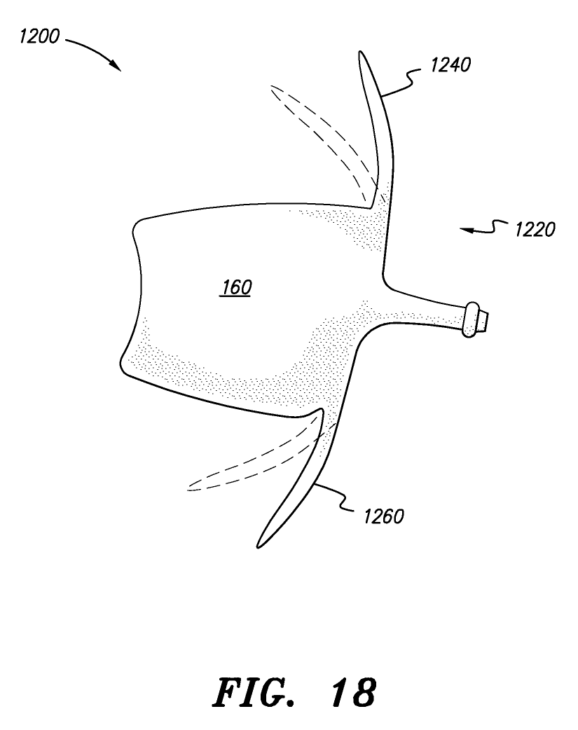

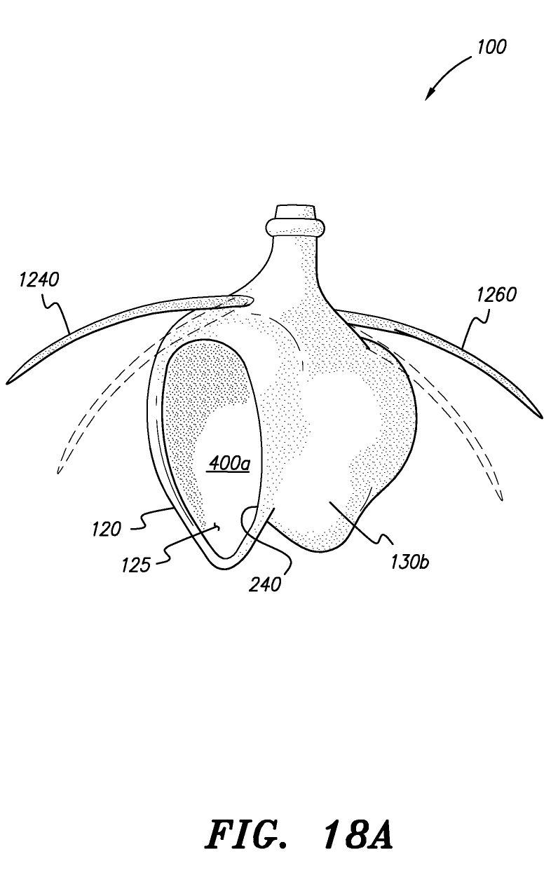

FIG. 18 shows another approach to the issue of securing a urine catchment device on the body, and illustrates a further example of a urine catchment device 1200. The urine catchment device 1200 shown in FIG. 18 comprises a spring 1220 made up of an upper spring spar 1240 and a complementary lower spring spar 1260, both attached to the body of the urine catchment device 1200, and extend outward from the bowl 120 as shown. The upper spring spar 1240 and lower spring spar 1260 are shown in their uncompressed positions in solid lines, and as deflected in broken lines. The uncompressed positions are those which would be seen when the spring 1220 is not compressed by the garment worn by the patient, such as the briefs B (see FIG. 6); for example, the spring spars 1240 and 1260 can be compressed in the vertical plane against the center portions of the front and rear sides of the briefs when the female user is in the supine position. Donning of such a garment would move the spring 1220 to the compressed or deflected positions indicated in broken line showings, such that resilient engagement of the garment is caused by spring action of the spring 1220 to maintain the device 100 in the operative position. The spring 1220 may be of the same constituent material as the rest of the urine catchment device 1240, or may comprise a different constituent material. For example, the spring 1220 may comprise a thin metallic leaf (not separately shown) embedded within a surrounding coating of the constituent material of the urine catchment device 1200.

The spring 1220 can also be molded into the urine catchment device 100; the urine catchment device 100 can also comprise the spring 1220, i.e., the bowl 120 can include upper and lower spring spars 1240 and 1260 as shown in FIG. 18A. The upper and lower spring spars 1240 and 1260 respectively extend upward and downward in the vertical plane at right angles with respect to the plane 400p of the opening 400a (see next paragraph).

The plane of the opening 400a defines an opening plane 400p that can be inclined in a downward direction towards end 380 from the end 385. The downward angle between the plane of the opening 400a and the horizontal can vary between 0° and 25°. Thus, the plane 400p can be horizontal or be inclined in a downward direction up to 25°; this is shown in FIG. 7A which shows a downward angle alpha between the plane 400p and the horizontal plane HP. It will be understood that the plane angle of the openings in the other embodiments described in this paper can also be horizontal (i.e., have an alpha angle of 0°) or can be inclined in a downward direction up to 25°.

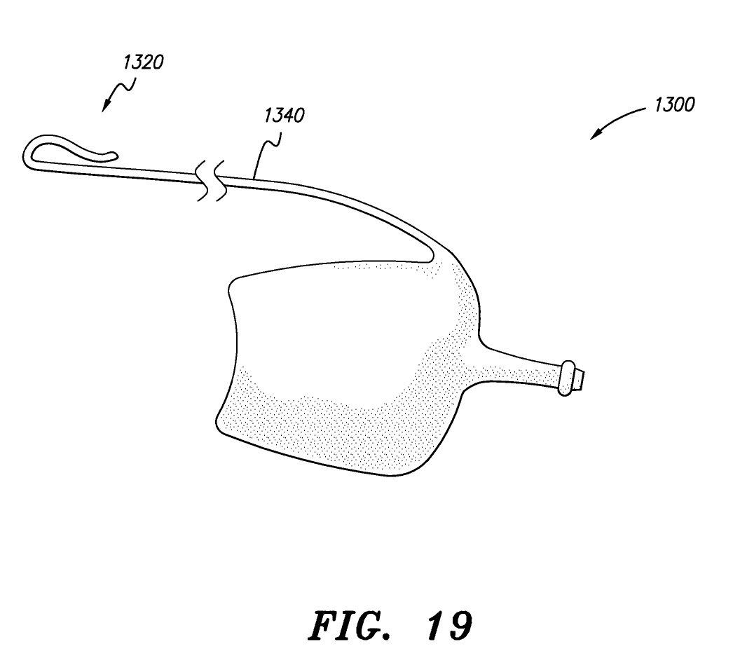

Still another way of engaging the garment is shown in FIG. 19. FIG. 19 depicts a further example of a urine catchment device 1300 which is structurally the same as the urine catchment device 100 except that the device 1300 further comprises a hook 1320 attached to the bowl 120 by means of an elongated hook connecting member 1340 as shown in FIG. 19. More specifically, the hook is attached to one end of the elongated hook connecting member and the other end of the elongated hook connecting member is attached to the bowl. In typical use, the hook 1320 is slipped over the edge of the briefs B (see FIG. 6) or other garment worn by the user of the urine catchment device 1300. A clip or other fastener (not shown) may be used in place of the hook 1320. The elongated member 1340 may be elastic and may have spring characteristics.

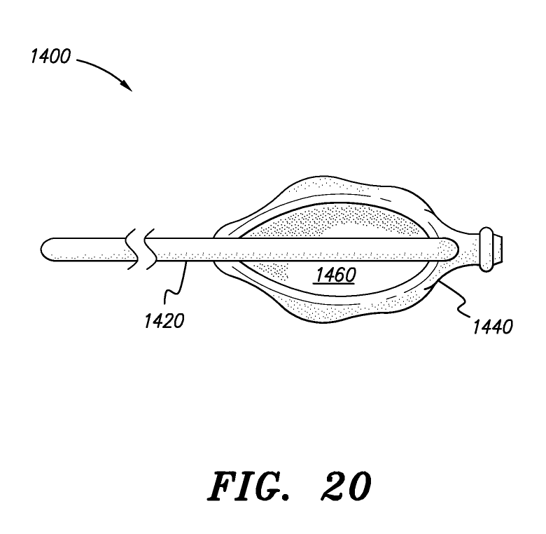

FIG. 20 shows another example of a urine catchment device 1400 which may be generally functionally and structurally similar to the urine catchment device 100 for example. The urine catchment device 1400 may incorporate a single member spring 1420 anchored at one end to the rear wall 1440 of the urine collection receptacle 1460. The spring 1420 may elastically bend downwardly against the urine collection receptacle 1460 as the garment such as the briefs B (see FIG. 6) compress thereover. Shape, curvature, width, thickness, and other characteristics of the spring 1420 or any corresponding spring utilized with the present invention may be varied in configuration or constituency or both to cooperate with anatomical detail, to optimize spring action, and to promote comfort.

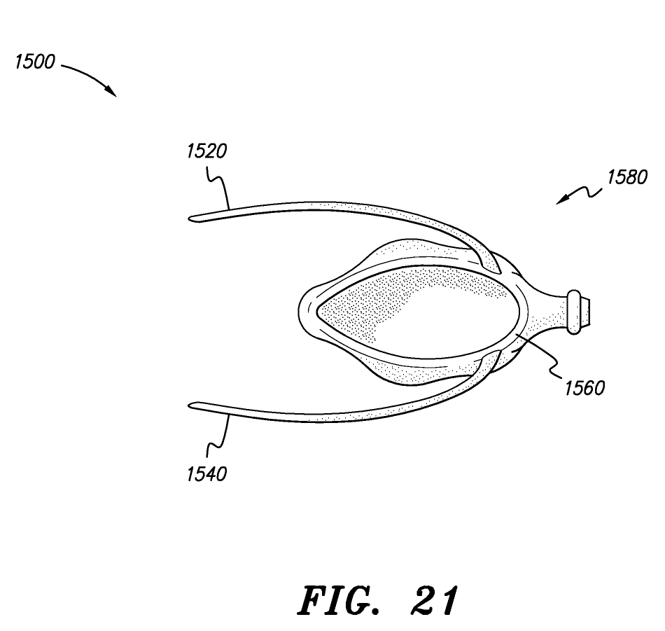

FIG. 21 shows a still further example of a urine catchment device 1500 which may be generally functionally and structurally similar to the urine catchment device 100 for example. The urine catchment device 1500 may incorporate a two member spring comprising a first spring member 1520 and a second spring member 1540. The spring members 1520 and 1540 may each be anchored at one end to the lip 1560 of the urine collection receptacle 1580. Alternatively, spring members such as the spring members 1520 and 1540 may be anchored to other parts of the urine catchment device 1500 or another urine catchment device according to the present invention.

The spring members 1520 and 1540 may elastically bend downwardly against the urine collection receptacle 1580 and/or the female user's body; wherein the female user's briefs can also cooperatively work to bend the spring members 1520 and 1540 over the urine collection receptacle 1580 and against the female user's body. It will be seen that the spring members 1520 and 1540 project in a direction towards the head of the patient when the urine catchment device 1500 is installed on the user's body in the manner depicted in FIGS. 5 and 6.

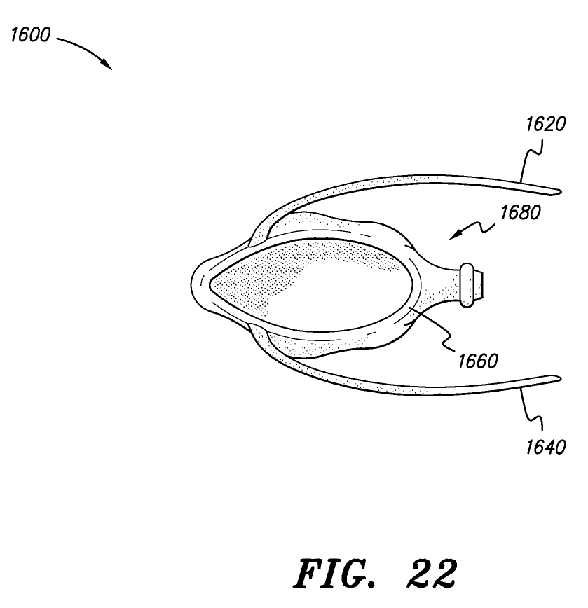

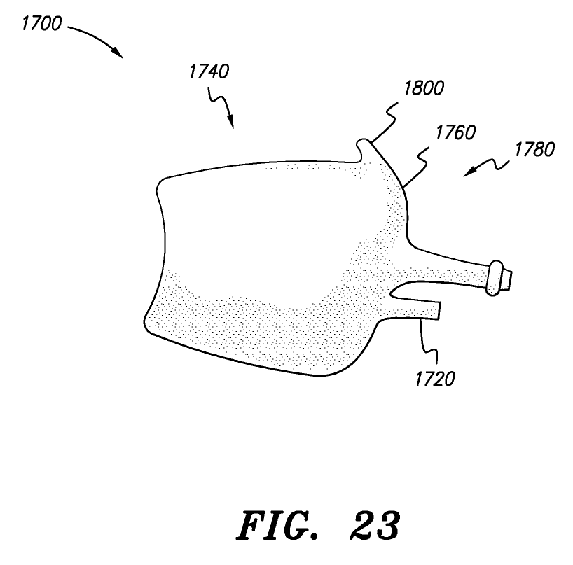

FIG. 22 shows a urine catchment device 1600 which is generally similar in structure and function to the urine catchment device 1500 of FIG. 21. However, in the urine catchment device 1600, spring members 1620 and 1640, which in other ways may be identical to corresponding spring members 1520 and 1540, are arranged to project in a direction facing the feet of the user rather than towards the head of the user.

It will be appreciated that the spring members 1520 and 1540 of FIG. 21 and the spring members 1620 and 1640 of FIG. 22 may be anchored at the side walls of their respective urine collection receptacles 1580 or 1680 or alternatively to the respective lips 1560 (FIG. 21) or 1660 (FIG. 22).

Spring members such as the spring members 1620 and 1640 may additionally be held in place by leg encircling members, such as elastic bands, garters, and the like (none shown).

FIG. 23 shows yet another example of a urine catchment device 1700 which may be generally functionally and structurally similar to the urine catchment device 100 for example. The urine catchment device 1700 may comprise a conduit stub 1720 which serves as a supply conduit for a genital rinsing or spraying appliance (not shown in its entirety). A supply of pressurized cleaning fluid (not shown) such as water may be connected to the conduit stub 1720. The conduit stub 1720 will be understood to have an internal passage (not shown) to deliver supplied cleaning fluid to the genital area, which is accessible from the urine collection receptacle 1740. The internal passage may incorporate a nozzle, spray head, or other device for directing flow (none of these is shown), which may be integral with the urine catchment device 1700 or which may be removable therefrom.

The urine catchment device 1700 may have a finger grip handle 1760 molded therein. The handle 1760 may take the form of two opposed recesses (not shown) formed in the rear wall 1780 of the urine collection receptacle 1740. The recesses may have individual recesses for receiving fingertips, or any other structure which is commonly utilized for handles and grips molded into bottles and other hand held containers (none shown).

The urine catchment device 1700 may have a short hood 1800 projecting from the rear wall 1780. The short hood 1800 may be adapted to provide at least some of the benefits of the relatively larger hood 1040 seen in FIG. 16.

A urine catchment device according to any aspect of the present invention, such as the urine catchment device 100, may have dimensions suitable for performance as described. Since the human body may assume different dimensions and configurations, the urine catchment device may be dimensioned and configured accordingly.

Any urine catchment device according to the present invention may be formed in more than one section. For example, a urine catchment device may be formed in two sections wherein one section may comprise the actual urine collection receptacle, and the other section could comprise a partially surrounding support which not only holds the urine collection receptacle, but also bears anatomy engaging features.

It should be understood that where feasible, features presented in the singular may be provided in the plural, the opposite also holding true. Also, where utilized herein, mention of “at least one” explicitly contemplates one, two, and numbers greater than two.

Any of the features of any of the urine catchment devices described herein may be combined with any other features, where feasible.

It is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

What is claimed:

1. A urine catchment device (100) for use by a female, comprising:
a bowl (120), the bowl (120) comprises a circumferential lateral wall (140), a floor (160) and a discharge tube (180),
wherein the circumferential lateral wall (140) defines a front bulge (260), a right side bulge (280), and a left side bulge (300), a concave front surface (320), the front bulge (260) being located near the floor (160),
wherein each of the front (260), right side (280), and left side (300) bulges are convex bulges,
wherein the bowl (120) has a hollow interior chamber (125) with an opening (400a),
wherein the opening (400a) is defined by a lip (240), the hollow interior chamber (125) being in communication with the discharge tube (180), and
wherein the concave front surface (320) is located between the front bulge (260) and the lip (240) to provide a wedge shaped front of the device (100).

2. The urine catchment device (100) according to claim 1 further comprising a hook (1320) and an elongated hook connecting member (1340), wherein the hook (1320) is attached to one end of the elongated hook connecting member (1340) and the other end of the elongated hook connecting member (1340) is attached to the bowl (1320).

3. The urine catchment device (100) according to claim 1, further comprising a first concave recess (340) and a second opposed concave recess (360) respectively located between the right side (280) and left side (300) bulges and the lip (240).

4. A urine catchment device (100) for use by a female, comprising:
a bowl (120), the bowl (120) comprises a circumferential lateral wall (140), a floor (160), a discharge tube (180), and a spring (1220),
wherein the bowl (120) has a hollow interior chamber (125) with an opening (400a), the opening (400a) being defined by a lip (240), the hollow interior chamber (125) being in communication with the discharge tube (180),
wherein the opening (400a) defines an opening plane (400p);
wherein the spring (1220) comprises an upper spring spar (1240) and a lower spring spar (1260), and
wherein the upper (1240) and lower (1260) spring spars respectively extend upward and downward in the vertical plane at right angles with respect to the plane (400p) of the opening (400a).

5. The urine catchment device (100) according to claim 4,
wherein the circumferential lateral wall (140) comprises opposite left (130a) and right (130b) sides, a front bulge (260), a right side bulge (280), and a left side bulge (300), and
wherein each of the front (260), right side (280), and left side (300) bulges are convex bulges.

6. A urine catchment device for use by a female patient, comprising:
a bowl, the bowl comprises a circumferential lateral wall, a floor and a discharge tube,
wherein the bowl has a hollow interior chamber located above the floor, the hollow interior chamber has an opening, the opening being defined by a lip,
wherein the hollow interior chamber is in communication with the discharge tube, and the hollow interior chamber is located above the floor, and
wherein the urine catchment device comprises an additional open hollow chamber located below the floor.

7. The urine catchment device according to claim 6,
wherein the circumferential lateral wall defines a front bulge, a right side bulge, and a left side bulge, and
wherein each of the front, right side, and left side bulges are convex bulges.
